SEQUENCE LISTING (8 sequences)

SEQ ID NO: 1 - DNA, 13329 bp, Homo sapien
gatccaccctctcttctcatctctgagccgggtgttcccaaacttccctcctggtctgttcatccaccaggctctgagggccagccctgcctggcaaggggggaaccaaggggccaactttagttttccagaagcctctgtccaggggaggagtcgacgtgaacgaatcggtcacacacacgcagaaaggtgccgctgctggggacctgtggggcgggggcggggcagaaggaaggtcccctgtttgggggaccctttattaaaaacaggcggcaagctgaggcgtccagctgagttcatcccaggccccaaagtaatcgcacggccaataagccctgcctaagatgaggacgggtgggtctggaccgaggccctggcgggagggagggtcctgggcgtgccaccagctctccggtcggaagcttctgcatgggccgtgccctgcgctgggagactcctgcccgggcagccttgctccaaggtcggctccacagagggtgcccgccctctcagccctggcctgtggcacctgcccacagccctttcttccctggatgcagtttttgccccctctgtgtcctcggctgcacgagtaggggctttcttgggttggctgcccgcctggcccggactgacccggactggctgaggctgaggctgacagtgcagggaaggagccagaagccactatggctgctgtgcagagaccaaggtgtctccctacacctgtggcccccagggccccagggacacagggtccacaccctgccccacctgctccatctccgggacgccctcgctccccaatctgattgcacagggtggggggcccagcagccttggtgacagttcttcatcccaagggcccgcccagtttctcctggctcctggggatgggagtggcctgggttgtggccccaccagctctgtgacagggctcttgatgcttctcaggccctagtttccctgagacctgctggccaggagctcaggcctcctggtttctggttacttttcctcccctagaaagcagccttggcagacagaacagaggcccagaagatcccgggaggctccccaggcccagaatctggggaacttgcaaggatttggaatcctggccgggtgtggtagccgagcctgtaatcccagaatttggggaggctaaggtgggaggattacttgaggccaggagattgagaccagcctgggcaacacagtgagaccccctctctacaaaaaaatttttaaaaatagccaggcgtgctggtgtgcccctgcagctccagctacttgggaggctgaaatgggaggatggcttgagcccgggaggtcaaggctgcagtgagccatgatcaagccactgcacttcagcctgggtgacaaagaccctgtctctcaaaataaacatttaaaaaatagaagttaaatcctcttttggagactgtggggtgaggggagtgtggccacaccacagcccttccacctccccattgtgtgccccgaactgtgctgtgctggccactggcctcaccctccctgaagcatggcaggtcccccacccccaaggccatgctggggtggggacaggggccatgtgcttcccacttggagggggctgttccagacacctccctggccgcccctggcagggtctcggctgtactggatgtgaggaccgtgggcctcccttcccccagactatgagagcctccaaaattgggaccgtgctgtttccctttccgtgctgtttcccaaagggcacccaggaaatgcttgctgcgtgaatcagtgaatgagtgagttcattcacctgggggctgggtggggacgatggagccttccagcctcctgggacctgccctcagtgtggaaagtgaggaggcatctgtcttcctgaggaaaacctgggcttagtcctccctctggcccaggaggggaccggaccccacagctggagggagccggcttagctgacagcgagtgtattaaaaacaagctttggagcaaagcggacaagctcaggtgttggtagagttcatcccaggccccaaagtaatcacatggcaaacaagccctgtctaaatatcacggcggctggggcagcggcacgcagcggcctggaaatgtcagccgggggtgggggctcctccgagccccgggattagcagaggtacctgaagtgaatgcgcccacctcctccttcctgctcctgctcaggacctgggctgggccagcccggggcacctggggaggggctcagagggtctcactggggccaggggctcttctttcagccccagcccgggctggttcccatggggtagcaggctgaggagagtggggagactgagcttggccggagtggggcggacgcacttccaggcccaaaccagcagcccacgggtgggggcagagaaagctgcccccctgcaggcccagtgagtcctcgagagagggggccacccggccatgggggggtggtgatgttggttcggggaacctagggcatctggaccagcccctggacaaggcggtcacagcagccactgcctgagcaggccacctgcgggcttccctccaggtctgcccatcggctcagggcttccagagcccaagggagcaacacgtttctcctgagcacgggtggagggaaaataaggatgtttacgatcgagttgcccatggaagcgttaccaagccccctggagactcatctcaccgcagtgggacctttgcattctctcaggcggtgggggtctctgccgtgttgtccgtaaagtgtcagcgtggggccaactggggacctcagcagccacgtccaaccctcatctgaaacaagaactggaggcctgggctgctcctcccttcccgccctcaggagcacagggtggcaggaggtgaactccatgggcgaggggctcttgctcttgcaggcccccaaagtcagtcaggtgcagaagggaaggacaggattcagggacaggagacacacagggggtcccctctgttccaggatgctcccaaatctgagcccagctgcccccagggtggagggtgcgtggacagccggccaagaggggcggggccacagaaggccctggcgaggccgtggggccaagcagaggagcctacagtggctggccagacgggtcctaggtgatgcaaggggtcctccgcacccctgttctgtttccccggctctgacccagtgtgcggcctctcctccatgtctgtatgtggctgcctccaaggcccctctcctcaggccctgtatgtccaagctgggcctccttcctctgatcgcccttgggagaggtggcattgaggtcacctcctcccctcccagagtctgcatcttgtgggcaaatgccccagtgcctcccaccatcctccatgcatgcagctgcctgcccaggtcccctgtgaacgcagcccagggccgtgcaggccacaggcggggctcatctccccaggtggggcctccaagtctacacctgtggctgggaaggggagtcacagcacagatggaatgaagcacatgagccctgggtgtggacctgcctcagctcagagcagcggtgggaccacatcttctccctgccacaggccaggtgactagcacccaagcccgtggcactggcactgctggggggccagggcgggctgtggccttgcaaggagatgtgatttgctgtcaaagcacagctgccgcctcggtgagtgactaatgagaactgaatgccgctcttattgcttttcactcgactaatttgtcagaggctgtcaagagccagggggagggggcagagggtggggaccggaggtctgattgagtcaccggcatgggggcgaggctgggtgcccggaggggtctgcaagaaaccaggagcacctggcaggaactcagggccggtggggaccttggccatgatgtcgtgtgagagtccggagggacacaggagctggggtcaccctgtttgttccatatcaatggctggtcagctcttctaagcccctactgtacacacacatgcacatgcatacataggacacacacacacacttacacaaacacacgcatgcatgcatgtggacataaaatcatacactcacatgaataattttagaagcatgtacaacacatgtacacgcagagaagcactcccacacatgcttcctggcacacacacacacacgcgcacacacacacacccttgaatgcacactctgtctcccacacagacacagaccagcgaaaactccaggccaagctctggtgcgtgggttcccaagcctggctgcacacacaaccagggtgctctcggcaattccagcatctccatacccctggagcctcttgtcctggtgtgggcttcctggtgatgtgggccagccaggtatgggtggaaccgtcctactccccctccagccccaagcctgagccagcctgagtctggcatggagctcctggagccaggtgagcagtgaggggcgctgggagctggggagatgccctgtgggtaggagatgcgcaccccgcccacccggatacccttcctcccagctgaatgcctggctgccagggaccacggtgacttctcttgcttggctctgtaacctgcccccttcgtaccctttccctccctctgcctccacctctgcccgactcggtcccacaggaccctctggccactggatccccttccctggaagcacccctcactgctcacctggctccaggagctccatgccagactcaggctggctcaggcttagggctggagggggagtaggacggttccacccatacctggctggcccacatcaccaggaagcccacaccaggacaagaggctccaggggtatggagatgctggaattgccgagagcaccctggttgtgtgtgcagccaggcttgggaacccacgcaccagagcttggcctggagttttcgctggtctgtgtctgtgggcctttgggggtcccacacacacaaggggctcaaggctgacccctcctcccacaagggcctgcaactgctaatccctgatgccccccactgtgtggatggcaaaactgagtccagggcccaaggggctgagtcaggaccctcttttcggccccctacatggtgggtctcaacactgaggcagtccctacaggcaacaaggatggaaggacagcactggctgtccaggctggagggactcagagaggaggccactgggggactgcctggaggaggagggcagcccgggcctgagggcctggcaggatttggtggggaagggaaagtggagccccaggtgggcagcagcagtagcagaaggggggcagggagccgtctgtgggggacagggagggtccggctgcctgtccagggtgtggaggaggagaggcagcccacaggctcagagcccgaaggaggcgtggtgcctgctctgccggcctcgctctgggcctgacttccaaacacccaattatccctaagtgcatccgatcgactggcagggcggctgttccggggcccacctcgtccatgcgctccgcccgccctgctgtggggctccatctgatggcctcattagggataattgctctggcatttgggtctgacagggacggcggattctgtcctgtgttggggcgtcttggttcttccagcttgggggatggaggggagctgcttccttacacggcagagaaaggccctgcaccccaggcggggcaagatggcgtgaggggaggatgcaggactcactgtcccctgccttcttgggacaatgggaactgagggacagcccagggtggcatgacaccccaaatcctcaggaggtcccccactgtctcccaaatgtgagtgggggtctgggaggctgcaggccggtgtccctgggagccaggctctagagggggcatctctggggaccctggggaccccgggctataaagagaactgcggagtagacatgggcgggggggcagtgtgtgctccagcatgtgtgtgtgtgtgtgcatgtacacgtgtgcacctgtatcgcctgtgtgtgtgcatgtgatgtgtacacgtgtcatgcatgcacgcacatgtgtagtgtgtgctcgtgtgtggtgtgtgcctgtgtcatgtatgagcacacttgtatatgttgtgtgtactgtgtcatatatgagtgtgtttgcctgtgtagtgcatgcacatccgtgtgtgcatctggtgtgtccgtgggtcattacgagtgcatcgtatgtgtatcgtgtacatgagtacacttgtatgtgtggtgtgtacaggtgccatgtaagtgtgcttgtacatatatgcatgcatgtgtcatatgcatctgtgtgtgcatgtgtgtggtgcacacatgtgttatgtctgagtgtgcctgtatgtgtgctatgtacacgtcatgtgtgagtgtgcttgcatgtgcagtgtgtggatgctgcttgtacctgtggtgtgtacctgtgtcatgggtgctcacacgtgcatggagtgttgtgtgtgtgcttgtgtgccccatgtgtgcatgtgtgtgtgcctcacacagatgcctgcatttgcctaggcacttgcaagaggacaccatgctggctctcaaagatcacagggccacctgagccctgtgcacaccacagccaggccatggctagaccctgcagagccacagggcgatgcctgtcagccaggggacccagaacacctcctgggcccctccccagcacatggctgggctcctccagcaggcctggatttgggaagggcccgtggtgggcaaggctggtgctggggagcaggcctggtggcctcagagactcgccctgtgggcggagcagcctcacagccaggttgaagtcagcactctgcccctgccccacgcggggagcgggcaccagtcccagggcacagacgtgctgggtgattaatctgggtgattaagcctcgggctgagaggctgttgagagagaacacgctccattgtggagctggctcagcattccttacggccatggtggcaggggctgtaaccacagggacggcggaagtggtggagggtggtggggtatggagggaagcccagagggctctgtgcaggaaggtggagcctggtgcaatggaggggacagcaagggctcctcagacctctgcggggcccccactcccctggtcacctgttttgtctctgatctggcctgggtcggccctcactcctggccccacctcatagccccccctggtggggctccgctccagcccttctccttcccaggggccagtatgctggccccaggggtctcttggggcgtgacctcggcctccagagaaccctgtcccagctctgcccttccctctggggtctctgtagatgggacgctggtcacagcagcctgtctgatttgttccctgtggcctaggttcctgagccccacagtgccaggggatggatgccaccggatctttgaaagaccagtgtcaggccgggcgcagtggctcacgcctgtaatcccagcactttgggaggccgaggtgggcggatcacgaagtcaggagatcgagaccatcctggctaacacggtgaaaccccgtctccactaaaaatacaaaaagttagctgggcgtggtggtgggcgcctgtagtcccagctactcgggaggctgaggcaggagaatggcgtgaaccggggaggcggagcttgcagtgagccgagatcgcgccattgcactccagcctgggtgacagagcgagactcggtctcaaaaaaaaaagaaaaaaaggaaagaccagtgtcttgggagttgggaaacctgggctggagactcactgcatgacccctgagaagttgcacctcagaacctcagtcctcgcatctgcagaatgggtctgtgaacacctcagctgcccgaacgtggatgccgcaggctgacccagcactgagctctaccaagaccaggggccagccgtgtgctccctccaggcctgtgcccagcgtggagaggcctcgtcccgtgggcgctggggtggagccttcctggtgtttgtggacatctctggagagggccagaggcaggtgggtgacacggggcatggctcaatcatgggtggtccagactggagaggtaccctcgggctgggagcggggaggctggccagggtggactttcggggcctccatggataccctcaccatctggaatcggagaggggcacggcacaaaggagggcggggccagggccaggactggagtcgggggcacctctgtgccaacaggggccttggatctggggtacagcatggttccccggccctgaaggggctggcgtgtgggacaggcttcccaggaatggataggcagggatggatgctgcctgattggggcgggaggctggaggcagggcaggtgcaggcacctgagggcagcactcacctccacaggggtccaggggcctccccagcctcagtacctggcctgggctcctgcctccagagagcctggccccaaggaagagtctagtaagcttagttcccatcgggcttccatgaaagcacaactggcccggcaggaaaccgaattaaaaagcaatatttgtatcagtggaagacatttgctgaaaggttaaatccacatccggcagtgtgggccatgagcctccggcgtggtgttcatcaggcatgtctctcctcctggcctgggcacctgagcactggggctgccctgggcagagctggggcagggtgctggggggcctggagctgcctcaccgagggatcctcagcagccgaccctgggggaggcaaatgagactctttctggggaccttgaggggagctcgggggagccatgcagagcttcaccaggcctggacactgggcatggaggctgggccacccaagggccatcaccagggactcaggtgggtgggcctcagccctgggtgacagaagctcacgggctgcagggcgaggccagaggctgagccttcaggctgaggtcttggaggcaaatccctccaacgcccttctgagcaggcacccagacctactgtgggcaggacccacaggaggtggaggcctttggggaacaccgtggaggggcatagcatctccgagagaggacagggtctgcactgggtgctgagagacagcaggggccgagcggtaggcttccctgcccccagggatgttccaggggagcgcaagggaggggcattaatatcgtggcaagaaagggcaggcattgcagagtgagcagcgacggaactgggttttgtgggatgcataggagttcacccggataagaggtgggtgaggaatgacactgcaaaccggggatcacggagccccaaatccttctgggccaggaagtgggaagggttggggggtcttccctttgctttgactgagcactcagcctgcctgcagagggcagcgaggagccacggaggggtgtgggacagggatgccatggctgaagcagttttaggaaaggtcccaggggctattgttgaagagagaacggggagcggggagtcccacagctgacaggagcagagtgggccctgagagatgccagctctgggtgccacagtgaccagccggggtaggccttcgagaagtcagggagcgtctagggcttctggctcctgctgggcccagggtgtcatcttgggctgccaacaccagaaagcccagcagatacaggaagccccaagccctgtcggaaacggttcttctccaggagggacagcggtggcagcgttcagccgcaggccatgcactctggggccacgtccttccctctgtacagtccagcattgtcaaggcgggctctggccatctctgctgaccccagagggatggggaggcctccccttccaccagaagggccagaagccaccctgggcaggggcatcactctccctgggtggggcagcggcggggagcaggaggtgccagtgggcgtgggctggatgcgggtgcctgcggggcggacatggaacttgggggaggctctaggctggggttgtcctcaagggagttctcaggtcaccccagggtcaccctcaacccggggcctggtggggtagaggagaaactgcaaaggtctctccaaggggaaggcatcagggccctcagcactgagggacgtgcgtgctcttcaaagaaggggccacaggaccccgagggaagccaggagctagcagtgggccatagaggggctgagtggggtgggtggaagccgtccctggccctggtcgccctggcaaccctggtggggactgtgatgcaggaggtggcagccatttggaaacgcgtggcgtctccttagagatgtcttcttcagcctcccagggtcctccacactggacaggtgggccctcctgggacattctggaccccacagggcgagcttgggaagccgctgcaagggccacacctgcagggcccgggggctgtgggcagatggcactcctaggaaccacgtctacaagacacacggcctggaatcttctggagaagcaaacaaattgcctcctgacatctgaggctggaggctggattccccgtcttggggctttctgggtcggtctgccacgaggttctggtgttcattaaaagtgtgcccctgggctgccagaaagcccctccctgtgtgctctcttgagggctgtggggccaaggggaccctggctgtctcagccccccgcagagcacgagcccctggtccccgcaagcccgcgggctgaggatgattcagacagggctggggagtgaaggcaattagattccacggacgagccctttctcctgcgcctccctccttcctcacccacccccgcctccatcaggcacagcaggcaggggtgggggatgtaaggaggggaaggtgggggacccagagggggctttgacgtcagctcagcttataagaggctgctgggccagggctgtggagacggagcccggacctccacactgagccatgcccacccccgacgccaccacgccacaggccaagggcttccgcagggccgtgtctgagctggacgccaagcaggcagaggccatcatggtaagagggcaggtaggtgcccggcggccgcagtggaccggagcccagggctggtgccagctgcctctgctactccccagcctggctggcagccccaggctcagggtccatgcaaacccctgggacgcggcgtggatgtggaggcctgggcacagcggcatcccctgtgcctggtgtttgagtccctgttgggggagggtgaggtgatgcctgtccctgtgtgtgcccctcttaggccgacctctctcgggggtcgtgtgggtctctgtgtcttgtttcatcttgaatcttaacgatcggaatgtggaaacaaatccatccaaaaaatccaagatggccagaggtccccggctgctgcacccagcccccaccctactcccacctgcccctgcctccctctgccccagctgccctagtcagcaccccaaccagcctgcctgcttggggaggcagccccaaggcccttcccaggctctagcagcagctcatggtggggggtcctgggcaaatagggggcaaaattcaaagggtatctgggctctggggtgattcccattggcctgttcctcccttatttccctcattcattcattcattcattcattcattcaccatggagtctgtgttccctgtgacctgcactcggaagccctgtgtacaggggactgtgtgggccaggctggataatcgggagcttttcagcccacaggaggggtcttcggtgcctccttgggcactcagaaccttgggctccctggcacatttaaaatgggtttttatttatggaccttgattgaaatgtggtgtgagttgtagcagtgtcatttccaggtaccttctcagggacacagggcgccctcccccgtcctcccccgccctcccctaccctcccccaccaggctccccatcaggcatcccctccccagggcgccccggggcccagcctcacaggctctccgtggcctggaactgcagccccagctgcatcctacacccccaccccaagggtaagtaagaggggactctgggaggggcttctgctgctccccttcatgttccacaaccctggaagctcaggatgaagctgattcttctcttacaaggggcccagagccttcttgggagttcagctccaagggatgagccccaggtgtctgccaagtccccctctgtccaggcctgggacggctctgggatcgaggggtcagaggcgctgagcccagggagagacacctgcgcccagagctatgacaaagggtggagggatgacaaggcagccaggagcgggcgcctgcggggtggcacagaggggcagggcccgaggacaggtgtcctgatgggagtgtgagaaagggtcccctgtgcggcagccaggagggtaggggggttgttcactggggccctgtgggggcagctccttcctgagctgccgttccctccccggcagccgatgccactgtccatcaagacatcgccctcttcccatcactaatccagttagcgcctggcctggggatgagtgacacagcgtctctgtctgtctgctcgccacagagtgggggagcaggcgagcaccttcccagcccccactcctcccccaccaccactgcttctgactgggctgcccccatcgggaagggcgtgcaatgcccgcaggcacctcggctagcatctgccccagcaggcacacagtaggcgctcaaaaacgtgctctcatcccctgcctctgtgtgccatcagcgctgcccgactgtgggaccagctgtgggtgggggtccccgggtctcagcaggtggaggaggcatgggtgccccttgtccccacagtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccatgctaaacctgctcttctccccgagggccaccaagccctcggcgctgtcccgagctgtgaaggtgtttgaggtgagctggtggccttcgtgtccctggggcaagttcacctgtgggtggggctgtgtgggctgagttcctgacccctctatagcagaggtgcagctgcccaggcccccgaggccggcacaggatgcagcaggggagtctcaggcctcagctcagcccccatggcatctagccacacccccgtgtttttgagggatc

SEQ ID NO: 2 - DNA, 10828 bp, Homo sapien
tcgacgtgaacgaatcggtcacacacacgcagaaaggtgccgctgctggggacctgtggggcgggggcggggcagaaggaaggtcccctgtttgggggaccctttattaaaaacaggcggcaagctgaggcgtccagctgagttcatcccaggccccaaagtaatcgcacggccaataagccctgcctaagatgaggacgggtgggtctggaccgaggccctggcgggagggagggtcctgggcgtgccaccagctctccggtcggaagcttctgcatgggccgtgccctgcgctgggagactcctgcccgggcagccttgctccaaggtcggctccacagagggtgcccgccctctcagccctggcctgtggcacctgcccacagccctttcttccctggatgcagtttttgccccctctgtgtcctcggctgcacgagtaggggctttcttgggttggctgcccgcctggcccggactgacccggactggctgaggctgaggctgacagtgcagggaaggagccagaagccactatggctgctgtgcagagaccaaggtgtctccctacacctgtggcccccagggccccagggacacagggtccacaccctgccccacctgctccatctccgggacgccctcgctccccaatctgattgcacagggtggggggcccagcagccttggtgacagttcttcatcccaagggcccgcccagtttctcctggctcctggggatgggagtggcctgggttgtggccccaccagctctgtgacagggctcttgatgcttctcaggccctagtttccctgagacctgctggccaggagctcaggcctcctggtttctggttacttttcctcccctagaaagcagccttggcagacagaacagaggcccagaagatcccgggaggctccccaggcccagaatctggggaacttgcaaggatttggaatcctggccgggtgtggtagccgagcctgtaatcccagaatttggggaggctaaggtgggaggattacttgaggccaggagattgagaccagcctgggcaacacagtgagaccccctctctacaaaaaaatttttaaaaatagccaggcgtgctggtgtgcccctgcagctccagctacttgggaggctgaaatgggaggatggcttgagcccgggaggtcaaggctgcagtgagccatgatcaagccactgcacttcagcctgggtgacaaagaccctgtctctcaaaataaacatttaaaaaatagaagttaaatcctcttttggagactgtggggtgaggggagtgtggccacaccacagcccttccacctccccattgtgtgccccgaactgtgctgtgctggccactggcctcaccctccctgaagcatggcaggtcccccacccccaaggccatgctggggtggggacaggggccatgtgcttcccacttggagggggctgttccagacacctccctggccgcccctggcagggtctcggctgtactggatgtgaggaccgtgggcctcccttcccccagactatgagagcctccaaaattgggaccgtgctgtttccctttccgtgctgtttcccaaagggcacccaggaaatgcttgctgcgtgaatcagtgaatgagtgagttcattcacctgggggctgggtggggacgatggagccttccagcctcctgggacctgccctcagtgtggaaagtgaggaggcatctgtcttcctgaggaaaacctgggcttagtcctccctctggcccaggaggggaccggaccccacagctggagggagccggcttagctgacagcgagtgtattaaaaacaagctttggagcaaagcggacaagctcaggtgttggtagagttcatcccaggccccaaagtaatcacatggcaaacaagccctgtctaaatatcacggcggctggggcagcggcacgcagcggcctggaaatgtcagccgggggtgggggctcctccgagccccgggattagcagaggtacctgaagtgaatgcgcccacctcctccttcctgctcctgctcaggacctgggctgggccagcccggggcacctggggaggggctcagagggtctcactggggccaggggctcttctttcagccccagcccgggctggttcccatggggtagcaggctgaggagagtggggagactgagcttggccggagtggggcggacgcacttccaggcccaaaccagcagcccacgggtgggggcagagaaagctgcccccctgcaggcccagtgagtcctcgagagagggggccacccggccatgggggggtggtgatgttggttcggggaacctagggcatctggaccagcccctggacaaggcggtcacagcagccactgcctgagcaggccacctgcgggcttccctccaggtctgcccatcggctcagggcttccagagcccaagggagcaacacgtttctcctgagcacgggtggagggaaaataaggatgtttacgatcgagttgcccatggaagcgttaccaagccccctggagactcatctcaccgcagtgggacctttgcattctctcaggcggtgggggtctctgccgtgttgtccgtaaagtgtcagcgtggggccaactggggacctcagcagccacgtccaaccctcatctgaaacaagaactggaggcctgggctgctcctcccttcccgccctcaggagcacagggtggcaggaggtgaactccatgggcgaggggctcttgctcttgcaggcccccaaagtcagtcaggtgcagaagggaaggacaggattcagggacaggagacacacagggggtcccctctgttccaggatgctcccaaatctgagcccagctgcccccagggtggagggtgcgtggacagccggccaagaggggcggggccacagaaggccctggcgaggccgtggggccaagcagaggagcctacagtggctggccagacgggtcctaggtgatgcaaggggtcctccgcacccctgttctgtttccccggctctgacccagtgtgcggcctctcctccatgtctgtatgtggctgcctccaaggcccctctcctcaggccctgtatgtccaagctgggcctccttcctctgatcgcccttgggagaggtggcattgaggtcacctcctcccctcccagagtctgcatcttgtgggcaaatgccccagtgcctcccaccatcctccatgcatgcagctgcctgcccaggtcccctgtgaacgcagcccagggccgtgcaggccacaggcggggctcatctccccaggtggggcctccaagtctacacctgtggctgggaaggggagtcacagcacagatggaatgaagcacatgagccctgggtgtggacctgcctcagctcagagcagcggtgggaccacatcttctccctgccacaggccaggtgactagcacccaagcccgtggcactggcactgctggggggccagggcgggctgtggccttgcaaggagatgtgatttgctgtcaaagcacagctgccgcctcggtgagtgactaatgagaactgaatgccgctcttattgcttttcactcgactaatttgtcagaggctgtcaagagccagggggagggggcagagggtggggaccggaggtctgattgagtcaccggcatgggggcgaggctgggtgcccggaggggtctgcaagaaaccaggagcacctggcaggaactcagggccggtggggaccttggccatgatgtcgtgtgagagtccggagggacacaggagctggggtcaccctgtttgttccatatcaatggctggtcagctcttctaagcccctactgtacacacacatgcacatgcatacataggacacacacacacacttacacaaacacacgcatgcatgcatgtggacataaaatcatacactcacatgaataattttagaagcatgtacaacacatgtacacgcagagaagcactcccacacatgcttcctggcacacacacacacacgcgcacacacacacacccttgaatgcacactctgtctcccacacagacacagaccagcgaaaactccaggccaagctctggtgcgtgggttcccaagcctggctgcacacacaaccagggtgctctcggcaattccagcatctccatacccctggagcctcttgtcctggtgtgggcttcctggtgatgtgggccagccaggtatgggtggaaccgtcctactccccctccagccccaagcctgagccagcctgagtctggcatggagctcctggagccaggtgagcagtgaggggcgctgggagctggggagatgccctgtgggtaggagatgcgcaccccgcccacccggatacccttcctcccagctgaatgcctggctgccagggaccacggtgacttctcttgcttggctctgtaacctgcccccttcgtaccctttccctccctctgcctccacctctgcccgactcggtcccacaggaccctctggccactggatccccttccctggaagcacccctcactgctcacctggctccaggagctccatgccagactcaggctggctcaggcttagggctggagggggagtaggacggttccacccatacctggctggcccacatcaccaggaagcccacaccaggacaagaggctccaggggtatggagatgctggaattgccgagagcaccctggttgtgtgtgcagccaggcttgggaacccacgcaccagagcttggcctggagttttcgctggtctgtgtctgtgggcctttgggggtcccacacacacaaggggctcaaggctgacccctcctcccacaagggcctgcaactgctaatccctgatgccccccactgtgtggatggcaaaactgagtccagggcccaaggggctgagtcaggaccctcttttcggccccctacatggtgggtctcaacactgaggcagtccctacaggcaacaaggatggaaggacagcactggctgtccaggctggagggactcagagaggaggccactgggggactgcctggaggaggagggcagcccgggcctgagggcctggcaggatttggtggggaagggaaagtggagccccaggtgggcagcagcagtagcagaaggggggcagggagccgtctgtgggggacagggagggtccggctgcctgtccagggtgtggaggaggagaggcagcccacaggctcagagcccgaaggaggcgtggtgcctgctctgccggcctcgctctgggcctgacttccaaacacccaattatccctaagtgcatccgatcgactggcagggcggctgttccggggcccacctcgtccatgcgctccgcccgccctgctgtggggctccatctgatggcctcattagggataattgctctggcatttgggtctgacagggacggcggattctgtcctgtgttggggcgtcttggttcttccagcttgggggatggaggggagctgcttccttacacggcagagaaaggccctgcaccccaggcggggcaagatggcgtgaggggaggatgcaggactcactgtcccctgccttcttgggacaatgggaactgagggacagcccagggtggcatgacaccccaaatcctcaggaggtcccccactgtctcccaaatgtgagtgggggtctgggaggctgcaggccggtgtccctgggagccaggctctagagggggcatctctggggaccctggggaccccgggctataaagagaactgcggagtagacatgggcgggggggcagtgtgtgctccagcatgtgtgtgtgtgtgtgcatgtacacgtgtgcacctgtatcgcctgtgtgtgtgcatgtgatgtgtacacgtgtcatgcatgcacgcacatgtgtagtgtgtgctcgtgtgtggtgtgtgcctgtgtcatgtatgagcacacttgtatatgttgtgtgtactgtgtcatatatgagtgtgtttgcctgtgtagtgcatgcacatccgtgtgtgcatctggtgtgtccgtgggtcattacgagtgcatcgtatgtgtatcgtgtacatgagtacacttgtatgtgtggtgtgtacaggtgccatgtaagtgtgcttgtacatatatgcatgcatgtgtcatatgcatctgtgtgtgcatgtgtgtggtgcacacatgtgttatgtctgagtgtgcctgtatgtgtgctatgtacacgtcatgtgtgagtgtgcttgcatgtgcagtgtgtggatgctgcttgtacctgtggtgtgtacctgtgtcatgggtgctcacacgtgcatggagtgttgtgtgtgtgcttgtgtgccccatgtgtgcatgtgtgtgtgcctcacacagatgcctgcatttgcctaggcacttgcaagaggacaccatgctggctctcaaagatcacagggccacctgagccctgtgcacaccacagccaggccatggctagaccctgcagagccacagggcgatgcctgtcagccaggggacccagaacacctcctgggcccctccccagcacatggctgggctcctccagcaggcctggatttgggaagggcccgtggtgggcaaggctggtgctggggagcaggcctggtggcctcagagactcgccctgtgggcggagcagcctcacagccaggttgaagtcagcactctgcccctgccccacgcggggagcgggcaccagtcccagggcacagacgtgctgggtgattaatctgggtgattaagcctcgggctgagaggctgttgagagagaacacgctccattgtggagctggctcagcattccttacggccatggtggcaggggctgtaaccacagggacggcggaagtggtggagggtggtggggtatggagggaagcccagagggctctgtgcaggaaggtggagcctggtgcaatggaggggacagcaagggctcctcagacctctgcggggcccccactcccctggtcacctgttttgtctctgatctggcctgggtcggccctcactcctggccccacctcatagccccccctggtggggctccgctccagcccttctccttcccaggggccagtatgctggccccaggggtctcttggggcgtgacctcggcctccagagaaccctgtcccagctctgcccttccctctggggtctctgtagatgggacgctggtcacagcagcctgtctgatttgttccctgtggcctaggttcctgagccccacagtgccaggggatggatgccaccggatctttgaaagaccagtgtcaggccgggcgcagtggctcacgcctgtaatcccagcactttgggaggccgaggtgggcggatcacgaagtcaggagatcgagaccatcctggctaacacggtgaaaccccgtctccactaaaaatacaaaaagttagctgggcgtggtggtgggcgcctgtagtcccagctactcgggaggctgaggcaggagaatggcgtgaaccggggaggcggagcttgcagtgagccgagatcgcgccattgcactccagcctgggtgacagagcgagactcggtctcaaaaaaaaaagaaaaaaaggaaagaccagtgtcttgggagttgggaaacctgggctggagactcactgcatgacccctgagaagttgcacctcagaacctcagtcctcgcatctgcagaatgggtctgtgaacacctcagctgcccgaacgtggatgccgcaggctgacccagcactgagctctaccaagaccaggggccagccgtgtgctccctccaggcctgtgcccagcgtggagaggcctcgtcccgtgggcgctggggtggagccttcctggtgtttgtggacatctctggagagggccagaggcaggtgggtgacacggggcatggctcaatcatgggtggtccagactggagaggtaccctcgggctgggagcggggaggctggccagggtggactttcggggcctccatggataccctcaccatctggaatcggagaggggcacggcacaaaggagggcggggccagggccaggactggagtcgggggcacctctgtgccaacaggggccttggatctggggtacagcatggttccccggccctgaaggggctggcgtgtgggacaggcttcccaggaatggataggcagggatggatgctgcctgattggggcgggaggctggaggcagggcaggtgcaggcacctgagggcagcactcacctccacaggggtccaggggcctccccagcctcagtacctggcctgggctcctgcctccagagagcctggccccaaggaagagtctagtaagcttagttcccatcgggcttccatgaaagcacaactggcccggcaggaaaccgaattaaaaagcaatatttgtatcagtggaagacatttgctgaaaggttaaatccacatccggcagtgtgggccatgagcctccggcgtggtgttcatcaggcatgtctctcctcctggcctgggcacctgagcactggggctgccctgggcagagctggggcagggtgctggggggcctggagctgcctcaccgagggatcctcagcagccgaccctgggggaggcaaatgagactctttctggggaccttgaggggagctcgggggagccatgcagagcttcaccaggcctggacactgggcatggaggctgggccacccaagggccatcaccagggactcaggtgggtgggcctcagccctgggtgacagaagctcacgggctgcagggcgaggccagaggctgagccttcaggctgaggtcttggaggcaaatccctccaacgcccttctgagcaggcacccagacctactgtgggcaggacccacaggaggtggaggcctttggggaacaccgtggaggggcatagcatctccgagagaggacagggtctgcactgggtgctgagagacagcaggggccgagcggtaggcttccctgcccccagggatgttccaggggagcgcaagggaggggcattaatatcgtggcaagaaagggcaggcattgcagagtgagcagcgacggaactgggttttgtgggatgcataggagttcacccggataagaggtgggtgaggaatgacactgcaaaccggggatcacggagccccaaatccttctgggccaggaagtgggaagggttggggggtcttccctttgctttgactgagcactcagcctgcctgcagagggcagcgaggagccacggaggggtgtgggacagggatgccatggctgaagcagttttaggaaaggtcccaggggctattgttgaagagagaacggggagcggggagtcccacagctgacaggagcagagtgggccctgagagatgccagctctgggtgccacagtgaccagccggggtaggccttcgagaagtcagggagcgtctagggcttctggctcctgctgggcccagggtgtcatcttgggctgccaacaccagaaagcccagcagatacaggaagccccaagccctgtcggaaacggttcttctccaggagggacagcggtggcagcgttcagccgcaggccatgcactctggggccacgtccttccctctgtacagtccagcattgtcaaggcgggctctggccatctctgctgaccccagagggatggggaggcctccccttccaccagaagggccagaagccaccctgggcaggggcatcactctccctgggtggggcagcggcggggagcaggaggtgccagtgggcgtgggctggatgcgggtgcctgcggggcggacatggaacttgggggaggctctaggctggggttgtcctcaagggagttctcaggtcaccccagggtcaccctcaacccggggcctggtggggtagaggagaaactgcaaaggtctctccaaggggaaggcatcagggccctcagcactgagggacgtgcgtgctcttcaaagaaggggccacaggaccccgagggaagccaggagctagcagtgggccatagaggggctgagtggggtgggtggaagccgtccctggccctggtcgccctggcaaccctggtggggactgtgatgcaggaggtggcagccatttggaaacgcgtggcgtctccttagagatgtcttcttcagcctcccagggtcctccacactggacaggtgggccctcctgggacattctggaccccacagggcgagcttgggaagccgctgcaagggccacacctgcagggcccgggggctgtgggcagatggcactcctaggaaccacgtctacaagacacacggcctggaatcttctggagaagcaaacaaattgcctcctgacatctgaggctggaggctggattccccgtcttggggctttctgggtcggtctgccacgaggttctggtgttcattaaaagtgtgcccctgggctgccagaaagcccctccctgtgtgctctcttgagggctgtggggccaaggggaccctggctgtctcagccccccgcagagcacgagcccctggtccccgcaagcccgcgggctgaggatgattcagacagggctggggagtgaaggcaattagattccacggacgagccctttctcctgcgcctccctccttcctcacccacccccgcctccatcaggcacagcaggcaggggtgggggatgtaaggaggggaaggtgggggacccagagggggctttgacgtcagctcagcttataagaggctgctgggccagggctgtggagacggagcccgga

SEQ ID NO: 3 - DNA, 27 bp, Artificial Sequence
  PCR primer
gacagatctccgggctccgtctccaca

SEQ ID NO: 4 - DNA, 13 bp, Homo sapien
agacggagcccgg

SEQ ID NO: 5 - DNA, 10 bp, Homo sapien
atgcccaccc

SEQ ID NO: 6 - DNA, 38 bp, Homo sapien
tgtggagacggagcccggacctccacactgagccatgc

SEQ ID NO: 7 - DNA, 27 bp, Artificial Sequence
  PCR primer
ctgtctagaggcccgaggcagaggtgt

SEQ ID NO: 8 - DNA, 19 bp, Homo sapien
tgtggagacggagcccgga

(19) United States
(12) Patent Application Publication
(10) Pub. No.: US 2002/0106794 A1
(43) Pub. Date: Aug. 8, 2002

(54) Tyrosine hydroxylase 5' control elements and uses thereof

(76) Inventors: Lorraine Iacovitti, Gwynedd Valley, PA (US); Mark A. Kessler, Philadelphia, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 09/942,325

(22) Filed: Aug. 29, 2001

Related U.S. Application Data
(60) Provisional application No. 60/228,931, filed on Aug. 30, 2000.

(51) Int. Cl. A61K 48/00; C12N 5/08; C07H 21/04; C12N 15/87

(52) U.S. Cl. 435/368; 536/23.2; 435/455; 424/93.21

(57) ABSTRACT

The differentiated cells of the adult mammalian central nervous system (CNS) have little or no ability to generate new nerve cells. This inability to produce new nerve cells is a distinct disadvantage when the need to replace lost neurons arises due to injury or disease. The present invention provides the sequence of 10.828 kB of the human tyrosine hydroxylase promoter. This sequence is used to purify dopaminergic cells, thus providing treatment for neurological diseases or disorders, such as Parkinson's disease, wherein a biologically active tyrosine hydroxylase is limiting or absent.

### CROSS REFERENCE TO RELATED APPLICATIONS

This application claims priority, in part, under 35 U.S.C. §119 based upon Provisional Application No. 60/228,931 filed Aug. 30, 2000.

### FIELD OF THE INVENTION

The present invention relates to the fields of molecular biology and neurology, and to the identification and characterization of the promoter region of the human tyrosine hydroxylase gene and, more particularly, to a method of inducing cells to express the phenotype of dopaminergic cells.

### BACKGROUND OF THE INVENTION

Unlike many other cells found in different tissues, the differentiated cells of the adult mammalian central nervous system (CNS) have little or no ability to enter the mitotic cycle and generate new nerve cells. Neurogenesis, the generation of new neurons, is complete early in the postnatal period. The synaptic connections involved in neural circuits, however, are continuously altered throughout the life of the individual, due to synaptic plasticity and cell death. Although this inability to produce new nerve cells in most mammals (especially primates) may be advantageous for long-term memory retention, it is a distinct disadvantage when the need to replace lost neuronal cells arises due to injury or disease.

A central goal in Neurobiology has been the discovery of ways in which to either rescue dopamine (DA) neurons from the progressive degeneration that occurs in, for example, Parkinson's disease (PD) or replace lost tissue with transplanted cells capable of dopaminergic function. The latter strategy depends for its success on a reliable source of transplantable DA neurons and the identification of factors relevant to neuronal growth and survival. Unfortunately, progress on both of these fronts has been greatly impeded by the fact that DA neurons comprise <1-5% of the total cells currently found in fetal mesencephalic cultures or in transplants. Therefore, in recent years, the search for an unlimited source of homogeneous DA neurons has intensified. An object of the present invention is to provide highly enriched/pure DA neurons, either by isolating neurons from transgenic animals and/or by promoting differentiation of the appropriate traits in a self-renewing population of stem or precursor cells, thereby providing a source of cells that can be used to replace cells lost in neurological diseases or conditions.

One common neurological syndrome, Parkinsonism, has been the object of attempts at cell transplant therapy (1). Parkinsonism (a disease of unknown etiology) is caused by a loss of dopamine-producing neurons in the substantia nigra of the basal ganglia (2).

While the recent availability of human embryonic stem cells (3) holds great promise for the development of cell replacement therapy for a variety of diseases, the ability to direct a primitive cell along the desired developmental path is limited (4). Tyrosine hydroxylase (TH) is the rate-limiting enzyme in the synthesis of the neurotransmitter dopamine. Its expression is restricted anatomically to dopaminergic (DA) neurons. The loss of TH-expressing cells correlates with the loss of dopamine and the severity of Parkinsonian symptoms.

The present invention defines the human tyrosine hydroxylase (hTH) promoter sequence, thereby allowing for the engineering and selection of the dopaminergic phenotype. Thus, the invention disclosed herein provides a long sought, yet unfulfilled need, for the transplantation of specific nerve cells for the treatment of various neurological diseases or conditions, such as, but not limited to, psychosis, depression, Alzheimer's disease and Parkinson's disease. The present invention provides methods for the in vitro culture and proliferation of transfected or transgenic cells and for the use of these cells and their progeny as tissue grafts. The isolation and in vitro perpetuation of large numbers of these cells and their progeny are induced to differentiate, thereby allowing for their neurotransplantation, in the undifferentiated and/or differentiated state, into an animal to alleviate the symptoms of neurologic disease, neurodegeneration and central nervous system (CNS) trauma. These cells are further used for drug screening of putative therapeutic agents targeted to the nervous system.

### ABBREVIATIONS

“PD” means “Parkinson's Disease”

“CA” means catecholaminergic

“NSC” means “neural stem cells”

“CNS” means “central nervous system”

“PNS” menas “peripheral nervousl system”

“TH” means “tyrosine hydroxylase”

“DA” means “dopaminergic”

“EC” means “embryonal carcinoma”

“ES” means “embryonal stem”

“EG” means “embryonal germ cell”

“FACS” means “fluorescent activated cell sorting”

“hTH” means “human trysosine hydroxylase”

“EMSA” means “electrophoretic mobility shift assays”

“PCR” means “polymerase chain reaction”

“FGF” means “fibroblast growth factor”

“aFGF” means “acidic fibroblast growth factor”

“bFGF” means “basic fibroblast growth factor”

“EGF” means “epidermal growth factor”

“IGF” means “insulin-like growth factor”

“GDNF” means “glial-derived neurotrophic factor”

“EGFP” means “humanized green fluorescent protein”

“GFP” means “green fluorescent protein”

“FSC” means “forward scatter”

“SSC” means “side scatter”

“NSE” means “neuronal specific enolase”

“GFAP” means “glial fibrillary acidic protein”

“β-gal” means “β-galactosidase”

“nm” means “nanometer”

“6-OHDA” means “6-hydroxydopamine”

“BDNF” means “brain-derived neurotrophic factor”

“BMP” means “bone morphogenetic protein”

“PKA” means “protein kinase A”

“PKC” means “protein kinase C”

“DDC” menas “dopa decarboxylase”

“DBH” means “dopamine-B-hydroxylase”

“ChAT” means “choline acetyltransferase”

“GAD” means “glutamic acid decarboxylase”

“i.p.” means “intraperitonial”

“SN” means “substantia nigra”

“NGF” means “nerve growth factor”

“nt” means “nucleotide”

“ng” means “nanogram”

“ml” means “milliliter”

“bp” means “base pair”

“μM” means “micromolar”

“CMV” means “cytomegalovirus”

“GABA” means “γ-aminobutyric acid”

“HPLC-EC” means “high performance liquid chromotography-electrochemical”

“FUDR” means “flurodeoxyuridine”

“araC” means “cytosine arabinose”

“DM” means “1:1 DMEM:Ham's F12 (Life Technologies) supplemented with 1% ITS+ (Collaborative Biomedical), glucose (6 mg/ml), glutamine (204 ug/ml),and penicillin/streptomycin(100 U and 100 ug/ml, respectively).”

### DEFINITIONS

“animal” includes, but is not limited to, animals such as “cows, pigs, chickens, mammals, and humans”

“at or near a site of nerve damage” is meant to refer to the location where nerve cells are implanted in order to replace destroyed, damaged or dysfunctional nerve cells and/or to restore function resulting from destroyed, damaged or dysfunctional nerve cells. The location is defined as being a site where such implanted cells develop as replacement cells for destroyed, damaged or dysfunctional nerve cells and make the necessary linkages to restore function lost due to destroyed, damaged or dysfunctional nerve cells. In one embodiment, by “at or near the site of nerve damage” it is meant that cells are implanted not only at the site at which the nerve cells are actually damaged but also at sites caudal and rostral to the site of damage.

“cell” as used herein can be of neural or paraneural origin, including but not limited to fibroblasts, embryonic stem cells, embryonic germ cells, embryonal carcinoma cells, neural stem cells, or cell lines grown in vitro. Any cell that can be transplanted with the construct of the invention is encompassed by the present invention.

“reporter gene” includes, but is not limited to, β-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotrarisferase (HPH), thymidine kinase (TK), lacZ (encoding β-galactosidase), and xanthine guanine phosphoribosyltransferase (XGPRT). As with many of the standard procedures associated with the practice of the invention, skilled artisans will be aware of additional sequences that can serve the function of a marker or reporter. Thus, the list is merely meant to show examples of what can be used and is not meant to limit the invention.

“neurological disease or condition” can be Parkinson's disease, Alzheimer's disease, Huntington's disease, epilepsy, psychosis, depression, or traumatic brain injury. This list is not exclusive and other neurological diseases or conditions may benefit by the methods of the present invention, such neurological diseases or conditions are readily determined by those skilled in the art.

“cell lines” as used herein is meant to mean a single cell type permanently established cell culture which will proliferate indefinitely given appropriate fresh medium and space.

### DESCRIPTION OF THE DRAWINGS

FIG. 1. In 18 wells of a 24-well plate, SH-SY5Y human neuroblasotoma cells (ATCC cat. # CRL-2266) are transfected with a calcium coprecipitate of pRL-null (a promoter/enhancer minus control; Promega, Madison, Wis.) and pMAK 1150-5 (FIG. 2). Groups 1 and 3 are transfected with the reporter construct (luciferase, pMAK 1150-5). Groups 2 and 4 are transfected with the control construct (renilla, pRL-null). One day following transfection, the culture media is changed to DM for all groups and Bone Morphogenetic Protein-4 (250 ng/ml; R & D Systems) is added to the media of groups 3 and 4, and is ommitted from the media of groups 1 and 2. BMP-4 has previously been shown to specifically augment TH expression in the PNS (5-7) and CNS (8). Assays for luciferase and renilla luciferase (control) are performed 24 hours following the BMP-4 addition. The Dual-Luciferase Reporter Assay System (Promega, Part # TM040) is used to assay independently firefly luciferase (groups 1 and 3) and Renilla luciferase (groups 2 and 4) according to the manufacturer's instructions. The control reporter, pRL-null, does not significantly change with BMP-4 treatment (compare Group 2 to Group 4, p>0.5). In contrast, the hTH reporter construct has a significant increase in the relative light fluorescence (RLF) when treated with BMP-4 (compare Group 1 to Group 3, p<0.001; n=9). Thus, the transfected hTH promoter mimics the response to BMP-4 of cultured embryonic striatal neurons ex vivo (8).

FIG. 2. pMAK 1150-5. The unique sites (Bgl II, Afl II, Sal I and Not I) allow convenient insertion of reporters, introns, and poly(A) cassettes as directionally cloned Bgl II-Afl II fragments. Vector components can be exchanged as Sal I- Afl II or Not I-Aft II pieces.

### DESCRIPTION OF THE INVENTION

The 13.329 kb sequence, of the human tyrosine hydroxylase (hTH) gene is cloned and sequenced. Human tyrosine hydroxylase (hTH) promoter-reporter constructs utilizing a reporter gene, including, but not limited to: GFP, hrGFP (Stratagene),enhanced, humanized green fluorescent protein, EGFP (9-11) beta-lactamase/CCF2 (12),and luciferase (13) allow for the functional analysis of the hTH promoter in stable, transfected cells, such as, but not limited to, those derived from human embryonal carcinoma (EC) and human or murine embryonal stem (ES) cells, embryonal germ cells (EG), etc. Further, use of fluorescent reporters permits fluorescent activated cell sorting (FACS) of living cells, thus highly enriched/purified populations of dopaminergic (DA) cells are obtained for biochemical analysis.

Sequence analysis of 10.828 kb promoter region of the human tyrosine hydroxylase (hTH) gene

Expression of the single copy TH gene is subject to exquisite spatial and temporal regulation. Short-term response elements (CRE, AP-1) are located in the proximal several hundred base pairs of the 5′-flanking region (14,15). This fragmen, however, is insufficient to correctly specify reporter expression in transgenic mouse models (16). The distal 5′-flanking region contains essential enhancer and silencer functions. The present invention provides a human genomic clone encompassing exons 1, 2, and 3 (˜2.5 kb) plus 10.828 kb of 5′-flanking sequence (SEQ. ID. NO: 1 ). This 13,329 kb region (SEQ. ID. NO: 1) of the human sequence is sequenced and characterized.

A partial human tyrosine hydroxylase (hTH) cDNA is purchased from ATCC (ATCC 100604). An EcoRI-Xho I fragment of this cDNA is isolated and used to screen a commercially available lambda cDNA library purchased from Stratagene (catalog no. 936201). The longest hTH cDNA clone out of 2 million total plaques screened is isolated. The 5′ 350 base pairs (bp) extending from an internal Xho I site to an Eco RI site within the vector is purified for use as a hybridization probe. Of seven positive clones recovered by cre/lox mediated excision in plasmid pBSKS+, two contain identical (by restriction mapping) ˜15 kb inserts encompassing exons 1, 2, and 3 of the hTH gene and 11 kb of the 5′ flanking (promoter) region as determined by Southern blot analysis.

The hTH promoter is isolated from the lambda vector by agarose gel electrophoresis followed by electroelution. For “shotgun” sequencing this fragment is subjected to titrated ultrasonic shearing to produce an average fragment size of 1.5 kb. After treatment with mung bean nuclease and “polishing” with T4 DNA polymerase, the size range of 1.5-2 kb is isolated by electrophoresis followed by electroelution and the isolated fragments are ligated into Sma I-cut plasmid pBCKS (Stratagene). Isolated plasmids are sequenced using the big dye terminator polymerase chain reaction (PCR) sequencing method with ABI Prism apparatus and software. The resulting data is analyzed and assembled using Lasergene software (DNAstar, Madison, Wis.). Known sequence motifs and transcriptional response elements are identified using the TRANSFAC (Heidelberg) and tf.dat (GCG/Wisconsin) databases.

A subcloned SacII-KpnI fragment (1.168 kb) of SEQ. ID. NO: 1, including the transcription start site, is mutated by PCR using the T3 sequencing primer (pBSIISK-;Stratagene) and the oligonucleotide GACAGATCTCCGGGCTCCGTCTCCACA (SEQ. ID. NO: 3). The mutated sequence is isolated as an Aat II-Bgl II fragment and ligated to the larger 10.775 kb Sal I-Aat II 5′ promoter fragment (isolated in the cDNA library, supra) to yield the 10.828 kb Sal I- Bgl II sequence (SEQ. ID. NO: 2).

Promoter structure/function analysis and physical studies

As the present invention provides 13.329 kb of the hTH gene (SEQ. ID. NO: 1), the following regions are identified. The transcriptional start site is mapped to position 10,968 and comprises the sequence agacggagcccgg (SEQ. ID. NO: 4). Further, the translational start site is mapped to position 10,997 and comprises the sequence ATGCCCACCC (SEQ. ID. NO: 5). Moreover, the present invention identifies bicoid binding elements I-IV. Binding element I is located at position 1142-1150, and has the sequence GGGATTACA. Binding element II is located at 2161-2169, has the sequence GGGATTAGC and is found on the positive strand. Element III is located at position 5042-5050, and has the sequence GGGATTAGC. Element IV is located at position 7513-7521 and has the sequence GGGATTACA. Elements I, III, and IV are located on the minus strand. Further at position 8111-8119 on the minus strand is a binding element with the Gli consensus sequence gaccaccca, an important determinant of midbrain neuronal phenotype (17,18). Thus, the present invention provides the hTH promoter region wherein a number of putative response elements are identified.

Large scale deletions of the human tyrosine hydroxylase promoter are accomplished by serial exonuclease III digestion from the 5′ end of the promoter. An accurate complete sequence (infra) enables the design of constructs in which restriction fragments of the promoter are deleted. Point mutations of sequences indicated by functional studies or homology to known response elements are constructed by oligonucleotide-directed PCR mutagenesis, confirmed by sequence analysis, and reassembled as convenient restriction fragments of the promoter.

Nuclei and nuclear extracts are prepared from highly enriched/purified populations of cells (infra). Analysis of DNAse hypersensitive sites consists of titrated digestion of isolated nuclei, followed by Southern blotting using PCR-generated probes specific for different regions of the 5′-flanking region (19,20). At a higher level of resolution, DNAse footprinting (21,22) and electrophoretic mobility shift assays (EMSA) provide independent evidence of the interaction of specific sequences with DNA binding proteins (23-26). Commercially available antibodies are used in “supershift” assays to confirm the presence of known transcription factors in DNA/protein complexes. This comprehensive approach identifies previously undescribed DNA sequence response elements or motifs for which the corresponding binding protein is unknown. Several methods are available to clone transcription factors based on their capacity to bind to an oligomerized response element, including, but not limited to, the lambda gt 11 system (27), and the yeast one-hybrid system (28).

Induction of a DA phenotype

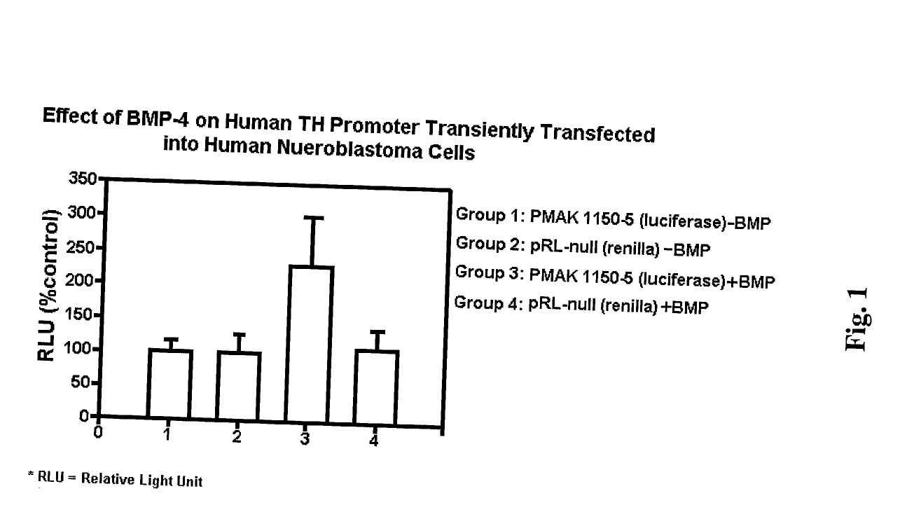

It has previously been shown that TH expression is induced and/or amplified in various cells (29-34). The synergistic interaction of specific cues initiates transcription of the normally quiescent TH gene in naive (non-TH-expressing) neurons in culture. FIG. 1 shows an example of such expression using a reporter gene (luciferase) to identify transcription initiation downstream of the hTH promoter (SEQ. ID. NO: 2) in human neuroblastoma cells. Thus, during a critical period in development just following withdrawal from mitosis, when newly differentiating neurons are phenotypically plastic, exposure to a specific growth factor, such as but not limited to, acidic fibroblast growth factor (aFGF), or to a lesser extent, basic FGF (bFGF) or brain-derived neurotrophic factor, bone morphogenetic protein 4 (BMP-4)(8), brain-derived neurotrophic factor (BDNF) (31,35) and a second obligatory co-activating molecule will trigger the novel expression of TH, as evidenced by TH immunocytochemistry and/or other detection assays (infra). Several sources of co-activators have been identified; including, but not limited to, brain and muscle extracts, CA neurotransmitters, and activators of the protein kinase A (PKA) and C (PKC) pathways (30-32,35,36).

The present invention provides vectors wherein the hTH promoter directs the expression of either TH, a therapeutic gene (infra), or a reporter gene. These constructs are transfected into cells, such as, but not limited to, EC, ES, EG, NSC, etc. Cells that have been transfected with the TH construct or the reporter gene construct are used in tissue culture to study the regulation of TH. Furthermore, cells that have been transfected with the hTH-reporter gene construct are used for enrichment of DA cells by FACS (infra). These cells may or may not require induction of the dopaminergic (DA) phenotype.

The present invention further provides cells that will substitute for missing DA neurons in animals. Thus, the TH expressing cells of the present invention represent the most highly enriched population of cells currently available for transplantation.

Isolation of TH expressing cells

Once TH expression is initiated in a cell such as, but not limited to EC ES, EG, neural stem cells, etc., it is stably expressed in vitro, even after differentiation cues have been removed (52). This has important implications for transplanting enriched/pure populations of these cells.

By transfecting cells with a fully expressing portion of the hTH promoter linked to a fluorescent reporter, it is possible to induce TH expression in vitro (and thus cause the cells to fluoresce), and then pass the cells through a FACS sorter and purify the fluorescent population of cells (i.e., cells which have been induced to different into DA neurons). This approach has been successfully demonstrated by FACS sorting of a variety of adherent and non-adherent rodent and human cells using GFP as a marker (37). Nagatsu and colleagues (16) reported that a 5 kb fragment of the human TH gene promoter directed strong expression of the reporter gene in all catecholaminergic tissues of adult transgenic mice. This construct, however, also produced ectopic expression. The present invention, therefore, provides a larger piece of the 5′ flanking region of the TH gene (10.828 kb; SEQ. ID. NO: 2) to control cell type specific expression of a downstream gene, such as TH, or other gene (such as a marker gene, growth hormone, neurotransmitter, therapeutic protein, etc).

Using the 10.828 kb of the human TH, a fusion within the 5′ untranslated region of the transcribed sequence is made to a reporter gene, such as, but not limited to, green fluorescent protein “GFP” (available in a promoterless vector, pEGFP-1 from Clontech positioned downstream of a multiple cloning site), luciferase, β-gal, etc.

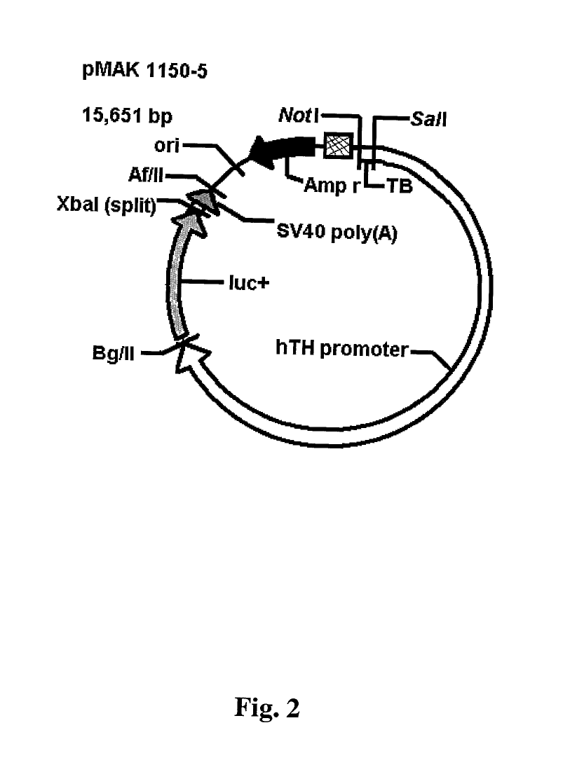

The present invention further provides a modified version of the hTH promoter sequence (SEQ. ID. NO: 2) wherein a unique Bgl II site is placed within the 5′ untranslated region (infra). This allows the isolation of the hTH promoter as a convenient 10.828 kb Sal II BGl II I cassette for use with a variety of vectors, as well as a variety of downstream genes, such as, but not limited to, reporter genes. The use of Bgl II does not introduce any unwanted modification(s) to the sequence of hTH promoter. The present invention provides a 10.828 kb hTH promoter sequence wherein this Bgl II is unique. pMAK 1150-5 (FIG. 2) is one embodiment of the present invention wherein unique sites allow convenient insertion of, for example, but not meant to limit the invention, reporters, introns, and poly(A) cassettes as directionally cloned Bgl II-Afl II fragments. Similarly, vector components are exchanged as Sal I- Afl II pieces. For all such manipulations it is understood that compatible cohesive ends (eg. BamHl-Bgl II, Xhol-Sal I, or Ava I-Sal I) are also employed as standard procedures of molecular biology.

Cells are transfected with the TH/reporter gene (for example luciferase) construct. Using G418 selection stable transfectants are cloned and expanded, some cells are differentiated, stimulated with TH-inducing agents and expressing cells are FACS sorted (supra).

Transplantation of cells expressing DA

Patients suffering from neurodegenerative diseases like Parkinson's are currently receiving transplants of unknown mixtures of developing midbrain cells (38-40). The present invention provides highly enriched/purified DA neurons, and when necessary, simultaneously with their trophic factors for transplantation and the treatment of neurodegenerative disorders.

The simultaneous treatment of highly enriched/purified DA neurons with growth factors improves survival after transplantation

It is possible that the host brain will adequately supply trophic support to transplanted neurons. If not, the present invention provides for the simultaneous infusions of specific growth substances onto DA neuron grafts to improve their prospects for survival.

The present invention provides a method in which to segregate DA neurons from other cell types (FACS sorting, supra). With highly enriched/pure DA neurons in hand, it is possible to transplant DA neurons. Thus, the present invention provides a rationale approach for developing therapeutic treatments for Parkinson's and other diseases involving compromised DA systems.

Transgenic animals

The development of transgenic animals has provided biological and medical scientists with models that are useful in the study of disease. Such transgenic animals are useful in testing pharmaceutical agents for utility in treating the disease as well as in testing of compounds that might cause or promote the development of such diseases. Such animals are also useful as sources of cells for tissue culture that can be used to study the causes of a particular disease.

The present invention provides transgenic animals wherein an hTH promoter construct (supra) is expressed. Such animals are useful as test subjects for determining the mutagenic potential of chemical compounds or agents. Further, the transgenic animals of the present invention are useful for providing a source of DA cells, following the isolation of TH expressing neurons, and possibly FACS enrichment for DA cells, for use in tissue culture studies and for transplantation (supra). Transgenic animals technology has also been used to study the tissue specificity of a cloned gene and expression of a transgene in vivo.

Methods for the generation of transgenic animals are well known to those of skill in the art (41-43). In accordance with the present invention, transgenic animals are developed through the introduction of a reporter gene under the control of the hTH promoter (SEQ. ID. NO: 2) into the germline of the mice. Such mice enable the functional analysis of hTH promoter to be carried out in vivo or ex vivo. For example, but not limited to, the mice of the present invention are used in drug discovery wherein various pharmaceutical agents are tested for their effect on the expression of the hTH promoter, thereby identifying potential therapeutic agents for the treatment of neurological diseases or conditions.

In accordance with another embodiment of the present invention, there are provided cells and cell lines derived from the above-described transgenic animals. The generation of cell lines derived from the above-described transgenic animals are readily accomplished by those of skill in the art. These transgenic cells of the present invention can be enriched using the reporter gene expression (i.e. fluorescence, etc.) to select and sort dopaminergic cells via FACS, these enriched cells are used for transplantation (infra) and tissue culture studies (infra) for in vitro analysis of the hTH promoter (supra).

EXPERIMENTAL METHODS

Standard techniques are used for recombinant nucleic acid methods, polynucleotide synthesis, cell culture, and transgene incorporation (e.g., electroporation, microinjection, lipofection). The enzymatic reactions, oligonucleotide synthesis, and purification steps are performed according to the manufacturer's specifications. The techniques and procedures are performed according to conventional methods in the art and various general references that are provided throughout this document. The procedures therein are well known in the art, some of which are provided for the convenience of the reader.

Modification of native human tyrosine hydroxylase
(hTH) promoter

| | |
|---|---|
| +1 +10 +20 +30 | |
| tgtggAGACGGAGCCCGGACCTCCACACTGAGCCATGC | (SEQ.ID.NO: 6) |
| tgtggAGACGGAGCCCGGAGATCTGTC | (SEQ.ID.NO: 7) |
| AGATCT | |
| Bgl ll * | |
| tgtggAGACGGAGCCCGGA | (SEQ.ID.NO: 8) |

Strategy used for modification of native human tyrosine hydroxylase (hTH) promoter. Only the “top” strand of a portion of the sequence is depicted for ease of alignment. Numbers at top characterize the proximal portion of the hTH transcript that begins with +1. Immediately below is the native hTH sequence with transcribed sequence in CAPS, and translated sequence in BOLD CAPS. Below this is SEQ. ID. NO: 7, the reverse complement of synthetic oligonucleotide MAKIL 124 (SEQ. ID. NO: 3) used for PCR-mediated mutation. In SEQ. ID. NO: 7, the font code is as described except that the mutating sequence is in BOLD ITALICS. Note that the mutation creates a Bgl II site (AGATCT) which after digestion (*) yields the final SEQ. ID. NO: 8. This unique synthetic Bgl II enables ligation to reporter sequence bearing a compatible cohesive end (e.g. Bgl II, BamHl, Bcl etc.)

Tissue Culture: Cultures of dopamine neurons are generated (supra) from cell lines or dissected fetal tissue. Methods are as described previously (36). In order to maximize yields, a method for microseeding is used. This is accomplished by plating cultures in a 25 μl drop from a micropipette tip in the center of each well in an 8-well Lab-Tek slide. Prior to plating, the culture chambers are coated with polyornithine (0.1 mg/ml), rinsed with H₂O and then left several minutes to evaporate off residual H₂O droplets. Surfaces treated in this manner permit the drop of cell suspension to stay positioned in the center 0.2 cm² of the well rather than seeping to the edge near the gasket. Thus, relatively few cells are used to plate many culture wells. Cell density is adjusted by increasing the concentration of cells in the drop without changing the volume of the drop (25 μl). Although cultures are seeded in a 10% fetal calf serum (FCS)-containing media, two hours after plating all cultures are rinsed and transferred into serum-free media (44).

TH Immunocytochemistry: Cultures are fixed in 4% paraformaldehyde and immunocytochemically stained for TH using the ABC/peroxidase method (VectaStain Elite Kit) as adapted for tissue culture (51). Stained cells are counted in 50% of all microscopic fields on the dish with the aid of an eyepiece reticle used at the 10× magnification.

TH Assay: In brief, prior to biochemical analysis, all cultures are rinsed in phosphate buffered saline (PBS; pH 7.2), scraped with a rubber policeman to free cells from the bottom of the well, and pelleted by low speed centrifugation (1000 RPM, 10 min, 40° C.). For enzyme assay, pelleted cells are disrupted by sonication in 5 mM potassium phosphate buffer (pH 7.0) containing 0.2% triton X-100 (v/v) at a dilution which insures that the reaction is linear with enzyme concentration and time. The sonicated cells are then centrifuged at 10,000×g for 10 min, and the supernatant decanted for assay of TH by conversion of ¹⁴C-labeled tyrosine to DOPA by the method of Coyle (45).

HPLC-EC: High pressure liquid chromatography (HPLC) with electrochemical detection (BAS 480 system) is used to quantify DA and its metabolites DOPAC and HVA. The HPLC technique is a standard one for quantifying tissue concentrations of catecholmines and metabolites (46). Catecholamines are quantified by peak height relative to standards after separation on 10 cm BAS Phase-2 ODS-reverse phase column using a pH 3.17 degassed phosphate buffer containing sodium octane sulfonate, methanol and EDTA as the mobile phase. Catecholamine levels are expressed on a per culture basis and are normalized to culture protein, content determined by the Lowry method. Means and standard errors for culture DA and catabolite concentrations, as well as metabolite/DA ratios, are calculated and compared between experimental groups of cultures.

DA Uptake: Since the amount of exogenous transmitter which can be taken up into nerve terminals varies with the number of terminal processes, DA uptake is a valuable index of dopamine innervation. Thus, it is possible with this method of analysis to assess whether growth actors are promoting the sprouting of more neuritic processes. Dopamine uptake is assessed in vitro by a modification (35) of the methods of Prochiantz et al. (47). Cultures are preincubated for 5 min at 37° C. with 250 μl of incubation solution (5 mM glucose and 1 mM ascorbic acid in PBS containing 1 mM pargyline. [³H]Dopamine (37 Ci/mmol) is then added to 50 nM and the cultures are incubated for another 15 min. Blanks are obtained by incubating cells at 0° C. Uptake is halted by removal of the incubation mixture, followed by 5 washes with cold PBS. The cultures are then lysed with 1% Triton X-100 with 10% perchloric acid and the ³H is measured by liquid scintillation counting.

Receptor Studies: Receptor plasticity can be a very important adaptive mechanism that occurs in response to its changing cellular/molecular milieu. Changes in dopamine receptor classes are examined using highly enriched/purified neurons grown with different cell types and in the presence of different growth factors. Procedures for radioligand binding studies are adapted from previously published studies done in culture (48). In brief, cultured cells are harvested, centrifuged twice at 500 g for 5 min at 20° C. in balanced salt solution. To obtain membranes, the pelleted cells are then lysed by flash-freezing in a dry ice/acetone bath. Lysed cells are slowly thawed, homogenized in 15 ml ice cold sucrose(265 nM) buffered with HEPES (50 mM, pH 7.4) using a teflon-glass homogenizer. Pooled supernatants are centrifuged at 37,000 g for 15 min. The protein contant of the membrane pellets is determined by the method of Peterson (49). Radioligand binding for D1 and D2 receptors using selective antagonists [³H]-SCH 23982 and [³H]-spiroperidol respectively, are assayed by incubating membranes as described in detail elsewhere (50). Radioactivity is monitored by liquid scintillation counting and saturation curves analyzed by Scatchard analysis.

Administration

According to some embodiments of the present invention, a sample from a culture of highly enriched/pure, stable, human neurons is transplanted into an individual being treated for a CNS injury, disease, condition or disorder. These cells replace and/or function in place of endogenous damaged, dead, non-functioning or dysfunctioning cells. Thus, in the case of an individual suffering from a CNS injury, disease, condition or disorder characterized by loss, damage or dysfunction of neurons such as, for example, diseases associated with nerve damage or spinal injury, the cells are transplanted into a site in the individual where the transplanted cells can function in place of the lost, damaged or dysfunctional cells and/or produce products needed to improve or restore normal functions that have been reduced or lost due to the lack of such products endogenously produced in the individual.

Methods for transplanting cells to specific regions of the central nervous system are taught by U.S. Pat. No. 5,650,148, incorporated herein by reference. These neural transplantation or “grafting” methods involve transplantation of cells into the central nervous system or into the ventricular cavities or subdurally onto the surface of a host brain.

The two main procedures for intraparenchymal transplantation are: 1) injecting the donor cells within the host brain parenchyma or 2) preparing a cavity by surgical means to expose the host brain parenchyma and then depositing the graft into the cavity. Both methods provide parenchymal apposition between the graft and host brain tissue at the time of grafting, and both facilitate anatomical integration between the graft and host brain tissue. This is of importance if it is required that the graft become an integral part of the host brain and to survive for the life of the host.

Alternatively, the graft may be placed in a ventricle, e.g. a cerebral ventricle or subdurally, i.e. on the surface of the host brain where it is separated from the host brain parenchyma by the intervening pia mater or arachnoid and pia mater. Grafting to the ventricle is accomplished by injection of the donor cells or by growing the cells in a substrate such as 3% collagen to form a plug of solid tissue, which plug is then implanted into the ventricle to prevent dislocation of the graft. For subdural grafting, the cells are injected around the surface of the brain after making a slit in the dura. Injections into selected regions of the host brain are made by drilling a hole and piercing the dura to permit the needle of a microsyringe to be inserted. The microsyringe is mounted in a stereotaxic frame and three dimensional stereotaxic coordinates are selected for placing the needle into the desired location of the brain or spinal cord.

Typically, the number of cells transplanted into the patient or host is a “therapeutically effective amount.” As used herein, “therapeutically effective amount” refers to the number of transplanted cells that are required to effect treatment of the particular disorder for which treatment is sought. For example, where the treatment is, for example, but not limited to, psychosis, depression, Alzheimer's disease and Parkinson's disease, transplantation of therapeutically effective amount of cells will typically produce a reduction in the amount and/or severity of the symptoms associated with the disease. Persons of skill in the art will understand how to determine proper cell dosages.

In some embodiments, it may be desired that the neurons be treated prior to transplantation in order to reduce the risk of stimulating host immunological response against the transplanted neurons. For example, in some embodiments, the cells are encapsulated by membranes prior to implantation. The encapsulation provides a barrier to the host's immune system and inhibits graft rejection and inflammation. It is contemplated that any of the many methods of cell encapsulation available are employed. In some instances, cells are individually encapsulated. In other instances, many cells are encapsulated within the same membrane. Several methods of cell encapsulation are well known in the art, such as those described in European Patent Publication No. 301,777 or U.S. Pat. Nos. 4,353,888; 4,744,933; 4,749,620; 4,814,274; 5,084,350; 5,089,272; 5,578,442; 5,639,275; and 5,676,943, each of which is incorporated herein by reference.

Injections of transplanted cells

Cells that are transfected with an hTH promoter construct (supra) are transplanted into a recipient host animal. In one embodiment, injections are made with sterilized 10 μl Hamilton syringes having 23-27 gauge needles. The syringe, loaded with cells, are mounted directly into the head of a stereotaxic frame. The injection needle is lowered to predetermined coordinates through small burr holes in the cranium. 40-50 μl of suspension are deposited at the rate of about 1-2 μl/min and a further 2-5 minutes are allowed for diffusion prior to slow retraction of the needle. If desired, multiple deposits are made along the same needle penetration. The injection is performed manually or by an infusion pump. At the completion of surgery following retraction of the needle, the host is removed from the frame and the wound is sutured. Prophylactic antibiotics or immunosuppressive therapy are administered as needed.

In other embodiments, the cells are transplanted into human patients. Patients travel to the hospital the day prior to surgery. The patient is admitted and examined by a medical professional either the night before or the day of surgery. A series of standard preoperative tests and a loading dose of phenytoin are given. Patients consume nothing by mouth after 10 p.m. the night before surgery.

During the surgery, a stereotactic surgical technique is performed using a CRW computed tomographic (CT) or magnetic resonance (MR) stereotaxic guide (Radionics, Burlington, Mass.). On the day of surgery, the stereotactic head ring is applied to the patient's head under local anesthesia. With the head ring in place, the patient undergoes CT or MR scanning. Baseline coordinates are established for the putamen. Typically, the long axis of the putamen is 30-40 mm in length, and with a height suitable for 2 needle passes on each side. Local anesthesia is used on the skin of the forehead. Incisions 1 cm in length are made in the skin. Implantation is carried out through two 3 mm twist drill holes in the forehead on each side of the midline, one above the other, both below the hairline, and both above the frontal sinus. The patient is awake but sedated with intravenously administered drugs such as midazolam.

All patients are admitted to the recovery room for postoperative observations. Postoperative CT or MR scans are taken to show evidence of hemorrhage. A follow-up appointment for suture removal is made at four to five days after surgery. All patients receive broad-spectrum antibiotics for three days. Phenytoin is administered as prophylaxis against seizures for three days after surgery.

In some embodiments, the concentration of cells delivered to the patient is 100-10⁷ cells/μl. In other embodiments, the concentration is 10³ to 10⁵ cells/μl. In yet other embodiments, the concentration is 10³ to 10⁴ cells/μl, with a total of 10⁷ cells delivered to the patient. Concentrations and doses, as well as site of transplantation (striatum, putamen, SN, etc.) may vary depending on the particular patient, neurological disorder, and other relevant factors. One skilled in the art is capable of determining the therapeutically effective amount appropriate under any given circumstances.

Pharmaceutical compositions

A pharmaceutical composition according to the present invention is useful for treating individuals suffering from injuries, diseases, conditions or disorders characterized by the loss, damage or dysfunction of endogenous neurons. A pharmaceutical composition is one that contains highly enriched/pure, stable, human neurons and a pharmaceutically acceptable medium.

The composition is formulated in accordance with routine procedures as a pharmaceutical composition adapted for intravenous administration to human beings. Typically, compositions for administration are solutions in sterile isotonic aqueous buffer. Where necessary, the composition may also include a solubilizing agent and a local anesthetic such as lidocaine to ease pain at the site of the injection. Where the composition is to be administered by infusion, it is dispensed with an infusion bottle containing sterile pharmaceutical grade saline. Where the composition is administered by injection, an ampoule of sterile saline for injection is provided so that the ingredients are mixed prior to administration.

Diagnostic uses

A hTH promoter polynucleotide and nucleic acids complementary thereto, and fragments thereof, are used for diagnostic purposes for disorders involving DA cells, as well as other disorders associated with TH expression. Such molecules are also used in diagnostic assays, such as immunoassays, to detect, prognose, diagnose, or monitor various conditions, diseases, and disorders associated with a mutation in the hTH promoter or monitor the treatment of disorders associated with TH, including, but not limited to psychosis (such as, but not limited to, schizophrenia bipolar disorder), depression, Alzheimer's disease and Parkinson's disease. Further, correlation of mutations in the hTH promoter are correlated with disease states for which it is a candidate gene, for example, but not limited to, schizophrenia bipolar disorder and other psychiatric illnesses. Accordingly, neurological disorders are diagnosed by detecting the presence of one or more mutations in the hTH promoter, alone or in combination with a decrease in expression of the TH transcript, in patient samples relative to TH expression in an analogous non-diseased sample. For diagnostic purposes, an hTH region polynucleotide is used to detect mutant TH gene expression in neurological diseases or conditions.

Polynucleotide sequences, and nucleic acids complementary thereto, of the hTH promoter, consisting of at least 8 to 25 nucleotides, are also useful as primers in primer dependent nucleic acid amplification methods for the detection of mutant hTH promoter sequences in patient samples. Primer dependent nucleic acid amplification methods useful in the present invention include, but are not limited to, PCR, competitive PCR, cyclic probe reaction, and ligase chain reaction. Such techniques are well known by those of skill in the art.

Expression systems

Methods which are well known to those skilled in the art are used to construct expression vectors containing a hTH promoter sequence and a gene of choice, such as, but not limited to, reporter genes (β-gal, luciferase, GFP, etc.), a therapeutic gene (NGF, neurotransmitter, growth hormone, etc.), or the TH gene in a DA neuron. These methods include in vitro recombinant DNA techniques, synthetic techniques and in vivo recombination/genetic recombination. See, for example, the techniques described in Sambrook et al., 2001, Molecular Cloning, A Laboratory Manual 3rd ed., Cold Spring Harbor Laboratory, N.Y. and Ausubel et al., 1989, Current Protocols in Molecular Biology, Greene Publishing Associates and Wiley Interscience, N.Y.

In one embodiment therapeutic agents are tested for their efficacy in modulating expression of the TH gene by monitoring the expression of a reporter gene. This allows for the development of alternative treatment regimens for neurological diseases or conditions relating to TH expression.

In one embodiment the expression of the downstream gene, for example a reporter gene, is used to isolate and purify DA neurons (supra). These cells provide highly enriched/pure cells for developmental/molecular studies, thus providing a system in which to better understand the growth and regeneration of neurons. Further, cells expressing a reporter gene are used to monitor the efficacy of cell transplantations.

In another embodiment the expression of a therapeutic gene expressing a neurologically-active compound in a DA neuron is provided. Examples of a therapeutic gene, not meant to limit the invention, include genes expressing a growth hormone (such as, but not limited to, NGF) to stimulate the development of neurons in a damaged region of the CNS; genes expressing a neurotransmitter to excite or inhibit a target neuron (such as, but not limited to, serotonin, dopamine, endorphins, etc.); or genes expressing a therapeutic protein for treatment of a disease or condition.

While this invention is described with a reference to specific embodiments, it is obvious to those of ordinary skill in the art that variations in these methods and compositions, such as the type of gene of interest to be expressed (supra), may be used and that it is intended that the invention may be practiced otherwise than as specifically described herein. Accordingly, this invention includes all modifications encompassed within the spirit and scope of the invention as defined by the claims.

### Reference List

1. Bjorklund, A. and Stenevi, U. (1979) Brain Research. 177, 555-560

2. McGeer, P. L., Itagaki, S., Akiyama, H., and McGeer, E. G. (1988) Annals.of.Neurology 24, 574-576

3. Thomson, J. A., Itskovitz-Eldor, J., Shapiro, S. S., Waknitz, M. A., Swiergiel, J. J., Marshall, V. S., and Jones, J. M. (1998) Science282, 1145-1147

4. Gage, F. H. (2000) Science 287, 1433-1438

5. Ernsberger, U. European.Journal.of.Biochemistry.267.(24.):6976.-6981., 2000.Dec. 6976-6981, 2000

6. Howard, M. J., Stanke, M., Schneider, C., Wu, X., and Rohrer, H. (9-1-0) Development. 127.(18.)A4073.-4081., 2000.Sep. 4073-4081, 2000

7. Reissmann, E., Ernsberger, U., Francis-West, P. H., Rueger, D., Brickell, P. M., and Rohrer, H. (1996) Development 122, 2079-2088

8. 8. Stull, N. D., Jung, J. W., and Iacovitti, L. (2001) Dev.Brain Research (in press)

9. Chalfie, M., Tu, Y., Euskirchen, G., Ward, W. W., and Prasher, D. C. (1994) Science 263, 802-805

10. Chalfie, M. (1995) Photochemistry.& Photobiology. 62, 651-656

11. Tsien, R. Y. (1998) Annual.Review.of.Biochemistry67, 509-544

12. Zlokarnik, G., Negulescu, P. A., Knapp, T. E., Mere, L., Burres, N., Feng, L., Whitney, M., Roemer, K., and Tsien, R. Y. (1998) Science 279, 84-88

13. Wood, K. V. (1995) Current.Opinion.in Biotechnology 6, 50-58

14. Guo, Z., Du, X., and Iacovitti, L. (1998) Journal of Neuroscience 18, 8163-8174

15. Liu, J., Merlie, J. P., Todd, R. D., and O'Malley, K. L. (1997) Brain Research.Molecular.Brain Research. 50, 33-42

16. Nagatsu, I., Karasawa, N., Yamada, K., Sakai, M., Fujii, T., Takeuchi, T., Arai, R., Kobayashi, K., and Nagatsu, T. (1994) Journal.of.Neural Transmission.-General.Section. 96, 85-104

17. Lee, J., Platt, K. A., Censullo, P., and Ruiz (1997) Development 124, 2537-2552

18. Sasaki, H., Hui, C., Nakafuku, M., and Kondoh, H. (1997) Development 124, 1313-1322

19. Forrester, W. C., Epner, E., Driscoll, M. C., Enver, T., Brice, M., Papayannopoulou, T., and Groudine, M. (1990) Genes & Development 4, 1637-1649

20. Tanaka, H., Zhao, Y., Wu, D., and Hersh, L. B. (1998) Journal.of.Neurochemistry. 70, 1799-1808

21. Carthew, R. W., Chodosh, L. A., and Sharp, P. A. (1985) Cell 43, 439-448

22. Tinti, C., Yang, C., Seo, H., Conti, B., Kim, C., Joh, T. H., and Kim, K. S. (1997) Journal.of.Biological.Chemistry. 272, 19158-19164

23. Fried, M. and Crothers, D. M. (1981) Nucleic.Acids.Research. 9, 6505-6525

24. Liang, C. L., Tsai, C. N., Chung, P. J., Chen, J. L., Sun, C. M., Chen, R. H., Hong, J. H., and Chang, Y. S. (6-26-1) Virology.277.(1.):184.-192., 2000.Nov. 10. 184-192, 2000

25. Schreiber, E., Matthias, P., Muller, M. M., and Schaffner, W. (1989) Nucleic.Acids.Research. 17, 6419

26. Wilson, D., Sheng, G., Lecuit, T., Dostatni, N., and Desplan, C. (1993) Genes & Development 7, 2120-2134

27. Vinson, C. R., LaMarco, K. L., Johnson, P. F., Landschulz, W. H., and McKnight, S. L. (1988) Genes & Development 2, 801-806

28. Gstaiger, M., Knoepfel, L., Georgiev, O., Schaffner, W., and Hovens, C. M. (1995) Nature 373, 360-362

29. Du, X., Stull, N. D., and Iacovitti, L. (1994) Journal of Neuroscience 14, 7688-7694

30. Du, X. and Iacovitti, L. (1995) Journal of Neuroscience 15, 5420-5427

31. Du, X., Stull, N. D., and Iacovitti, L. (1995) Brain Research 680, 229-233

32. Du, X. and Iacovitti, L. (1997) Journal of Neurochemistry 68, 564-569

33. Du, X. and Iacovitti, L. (1997) Brain Research.Molecular.Brain Research 50, 1-8

34. Iacovitti, L. (1994) Pharmacology & Therapeutics 62, 373-383

35. Knusel, B., Winslow, J. W., Rosenthal, A., Burton, L. E., Seid, D. P., Nikolics, K., and Hefti, F. (1991) Proceedings.of.the.National.Academy.of.Sciences.of.the.United.States.of.America. 88, 961-965

36. Iacovitti, L., Evinger, M. J., Joh, T. H., and Reis, D. J. (1989) Journal of Neuroscience 9, 3529-3537

37. Bierhuizen, M. F., Westerman, Y., Visser, T. P., Wognum, A. W., and Wagemaker, G. (1997) Biochemical.& Biophysical.Research.Communications. 234, 371-375

38. Freed, C. R., Breeze, R. E., Rosenberg, N. L., Schneck, S. A., Kriek, E., Qi, J. X., Lone, T., Zhang, Y. B., Snyder, J. A., and Wells, T. H. (1992) New England.Journal.of.Medicine 327,1549-1555

39. Spencer, D. D., Robbins, R. J., Naftolin, F., Marek, K. L., Vollmer, T., Leranth, C., Roth, R. H., Price, L. H., Gjedde, A., and Bunney, B. S. (1992) New England.Journal.of.Medicine 327, 1541-1548

40. Widner, H., Tetrud, J., Rehncrona, S., Snow, B., Brundin, P., Gustavii, B., Bjorklund, A., Lindvall, O., and Langston, J. W. (1992) New England.Journal.of.Medicine 327, 1556-1563

41. Brigid Hogan (1994) Manipulating the mouse embryo: a laboratory manual, Plainview, N.Y.: Cold Spring Harbor Laboratory Press,

42. Swanson, L. W., Simmons, D. M., Arriza, J., Hammer, R., Brinster, R., Rosenfeld, M. G., and Evans, R. M. (1985) Nature 317, 363-366

43. Wall, R. J., Pursel, V. G., Shamay, A., McKnight, R. A., Pittius, C. W., and Hennighausen, L. (1991) Proceedings.of.the.National.Academy.of.Sciences.of.the.United.States.of.America. 88, 1696-1700

44. Bottenstein, J. E. and Sato, G. H. (1979) Proceedings.of.the.National.Academy.of.Sciences.of.the.United.States.of.America. 76, 514-517

45. Coyle, J. T. (1972) Biochemical.Pharmacology 21, 1935-1944

46. Wagner, J., Vitali, P., Palfreyman, M. G., Zraika, M., and Huot, S. (1982) Journal.of.Neurochemistry. 38,1241-1254

47. Prochiantz, A., Daguet, M. C., Herbet, A., and Glowinski, J. (1981) Nature 293, 570-572

48. Ernsberger, P., Iacovitti, L., and Reis, D. J. (1990) Brain Research. 517, 202-208

49. Peterson, G. L. (1977) Analytical.Biochemistry 83, 346-356

50. Billard, W., Ruperto, V., Crosby, G., Iorio, L. C., and Barnett, A. (1984) Life Sciences. 35, 1885-1893

51. Iacovitti, L., (1991) J. Neurosci., 11:2403-2409

52. Iacovitti, L., Stull, N. D., and Jin H. (2001) Dev.Brain Research (in press)

1. A method of enriching/purifying DA cells away from non-DA cells, comprising:
a) transfecting cells with a fully expressing portion of a hTH promoter linked to a fluorescent reporter;
b) passing said cells from step a) through a flow cytometer;
c) separating fluorescent cells from non-fluorescent cells;
d) identifying hTH-expressing cells from non-expressing hTH cells; and
e) obtaining highly enriched/pure DA cells.

2. The method of claim 1 wherein said hTH promoter has a length of 1-13 kB.

3. The method of claim 2 wherein said hTH promoter, or fragment thereof, has a nucleotide sequence of SEQ. ID. NO: 1 or SEQ. ID. NO: 2.

4. The method of claim 1 or 2 wherein said transfected cells with said fully expressing portion of said hTH promoter linked to said fluorescent reporter are induced to differentiate prior to passing said cells from step a) through a flow cytometer.

5. An isolated nucleic acid comprising a sequence encoding a hTH promoter having the nucleotide sequence of SEQ. ID. NO: 1.

6. An isolated nucleic acid comprising a sequence encoding a hTH promoter having the nucleotide sequence of SEQ. ID. NO: 2.

7. An isolated nucleic acid that is capable of hybridizing to the hTH promoter sequence, said hTH promoter sequence having the sequence of SEQ. ID. NO: 2, said nucleic acid containing at least an 8 to 25 nucleotide portion of SEQ. ID NO: 2.

8. An isolated nucleic acid that is capable of hybridizing to the hTH promoter sequence, said hTH promoter sequence having the sequence of SEQ. ID. NO: 1, said nucleic acid containing at least an 8 to 25 nucleotide portion of SEQ. ID. NO: 1.

9. A method of diagnosing or screening for the presence of, or a predisposition for, developing a neurological disease or condition in an animal, comprising detecting one or more mutations in a hTH promoter DNA derived from said animal in which the presence of said one or more mutations indicates the presence of said neurological disease or disorder or a predisposition for developing said disease or disorder.

10. The method of claim 9 wherein said hTH promoter DNA is subjected to polymerase chain reaction using oligonucleotide primers adapted to amplify a fragment of said hTH promoter DNA.

11. A method of treating an animal having a neurological disease or condition characterized by a dopamine deficiency, comprising transplanting into said animal highly enriched/purified DA cells comprising a functional hTH promoter sequence directing expression of a biologically active tyrosine hydroxylase gene and wherein said DA cells are capable of expressing said tyrosine hydroxylase gene, and wherein a biologically active tyrosine hydroxlase translated from said tyrosine hydroxylase gene alleviates said neurological disease or disorder.

12. The method of claim 11 wherein said neurological disease or disorder is Parkinson's disease.

13. The method of claim 11 wherein said method further includes delivering at least one of aFGF, IBMX, forskolin, TPA, BMP or DA.

14. The method of claim 11 wherein said transplanting is into at least one of a central nervous system, a ventricular cavity, a dubdural surface of a brain, or a nigrostriatal system.

15. A method of treating an animal having a Parkinson's disease, comprising transplanting into at least one of a central nervous system, a ventricular cavity, a dubdural surface of a brain or a nigrostriatal system of said animal highly enriched/purified DA cells comprising a functional hTH promoter sequence directing expression of a biologically active tyrosine hydroxylase gene and wherein said DA cells are capable of expressing said tyrosine hydroxylase gene, and wherein a biologically active tyrosine hydroxlase translated from said tyrosine hydroxylase gene alleviates said Parkinson's disease.

16. A method for providing a neurologically-active compound to an animal, comprising transplanting into said animal a highly enriched/purified DA cell having been transfected with a vector containing a hTH promoter controlling the expression of a therapeutic gene encoding said neurologically-active compound and wherein said neurologically-active compound alleviates a disease or condition deficient in said neurologically-active compound.

17. The method of claim 16 wherein said therapeutic gene encodes a growth hormone or a neurotransmitter.

18. A pharmaceutical composition comprising isolated and highly enriched/purified DA cells having been transfected with a hTH promoter controlling an expression of a therapeutic gene and a pharamceutically acceptable carrier.

19. A pharmaceutical composition comprising isolated and highly enriched/purified DA cells and a pharamceutically acceptable carrier.

20. A method of diagnosing or screening for the presence of, or a predisposition for, developing a neurological disease or condition in an animal, comprising detecting one or more mutations in a hTH promoter DNA having a sequence of SEQ. ID. NO: 2 derived from said animal in which the presence of said one or more mutations indicates the presence of said neurological disease or disorder or a predisposition for developing said disease or disorder.

21. The method of claim 20 wherein said hTH promoter DNA having said sequence of SEQ. ID. NO: 2 is subjected to polymerase chain reaction using oligonucleotide primers adapted to amplify a fragment of said hTH promoter DNA having said sequence of SEQ. ID. NO: 2.

22. A method of treating an animal having a neurological disease or condition characterized by a dopamine deficiency, comprising transplanting into said animal highly enriched/purified DA cells comprising a functional hTH promoter sequence DNA having a sequence of SEQ. ID. NO: 2 directing expression of a biologically active tyrosine hydroxylase gene and wherein said DA cells are capable of expressing said tyrosine hydroxylase gene, and wherein a biologically active tyrosine hydroxlase translated from said tyrosine hydroxylase gene alleviates said neurological disease or disorder.

23. The method of claim 22 wherein said neurological disease or disorder is Parkinson's disease.

24. The method of claim 22 wherein said method further includes delivering at least one of aFGF, IBMX, forskolin, TPA, BMP or DA.

25. The method of claim 22 wherein said transplanting is into at least one of a central nervous system, a ventricular cavity, a dubdural surface of a brain or a nigrostriatal system.

26. A method of treating an animal having a neurological disease or condition characterized by a dopamine deficiency, comprising transplanting into said animal highly enriched/purified DA cells comprising a functional hTH promoter sequence DNA having a sequence of SEQ. ID. NO: 2 directing expression of a biologically active tyrosine hydroxylase gene and wherein said DA cells are induced to express said tyrosine hydroxylase gene prior to transplantation, and wherein a biologically active tyrosine hydroxlase translated from said tyrosine hydroxylase gene alleviates said neurological disease or disorder.

27. The method of claim 26 wherein said neurological disease or disorder is Parkinson's disease.

28. The method of claim 26 wherein said method further includes delivering at least one of aFGF, IBMX, forskolin, TPA, BMP or DA.

29. The method of claim 26 wherein said transplanting is into at least one of a central nervous system, a ventricular cavity, a dubdural surface of a brain or a nigrostriatal system.

30. A method for providing a neurologically-active compound to an animal, comprising transplanting into said animal a highly enriched/purified DA cell having been transfected with a vector containing a hTH promoter having a sequence of SEQ. ID. NO: 2 controlling the expression of a therapeutic gene encoding said neurologically-active compound and wherein said neurologically-active compound is alleviates a disease or condition deficient in said neurologically-acitve compound.

31. The method of claim 30 wherein said therapeutic gene encodes a growth hormone or a neurotransmitter.

32. A pharmaceutical composition comprising a pharamceutically acceptable carrier and an isolated and highly enriched/purified DA cells having been transfected with a hTH promoter having a sequence of SEQ. ID. NO: 2 controlling expression of a therapeutic gene.

33. A pharmaceutical composition comprising isolated and highly enriched/purified DA cells and a pharamceutically acceptable carrier.

34. A transgenic animal whose genome comprises a DNA construct comprising, in operable association, a hTH promoter and a DNA sequence encoding a reporter gene, wherein said hTH promoter has a length of up to 10.828 kb extending upstream from the transcription initiation site, and wherein said transgenic animal expresses said reporter gene such that dopaminergic neurons are identified.

35. The transgenic animal of claim 34 wherein said hTH promoter has the sequence of SEQ. ID. NO: 2.

36. The transgenic animal of claim 34 or 35 wherein said mouse is fertile and transmits said DNA construct comprising, in operable association, a hTH promoter and a DNA sequence encoding a reporter gene, to its offspring.

37. An isolated transgenic animal cell comprising a DNA construct comprising, in operable association, a hTH promoter and a DNA sequence encoding a reporter gene, wherein said hTH promoter has a length of up to 10.828 kb extending upstream from the transcription initiation site, and wherein said isolated transgenic animal cell expresses said reporter gene such that dopaminergic neurons are identified and highly enriched/purified away from non-dompaminergic neurons.

38. The isolated mouse cell of claim 37 wherein said hTH promoter has the sequence of SEQ. ID. NO: 2.

39. A DNA construct comprising, in operable association, a hTH promoter and a DNA sequence encoding a reporter gene, wherein said hTH promoter has a length of up to 10.828 kb extending upstream from the transcription initiation site.

40. The DNA construct of claim 39 wherein said hTH promoter has the sequence of SEQ. ID. NO: 2.

41. A DNA construct comprising, in operable association, hTH promoter and a DNA sequence encoding a therapeutic gene, wherein said hTH promoter has a length of up to 10.828 kb extending upstream from the transcription initiation site.

42. The DNA construct of claim 39 wherein said hTH promoter has the sequence of SEQ. ID. NO: 2.
